(19) United States
(12) Patent Application Publication
(10) Pub. No.: US 2022/0175799 A1
(43) Pub. Date: Jun. 9, 2022

(54) COMPOSITIONS HAVING THE ABILITY TO PROMOTE HEALTHY CHOLESTEROL LEVELS

(71) Applicant: Brilliant Lab LLC, American Fork, UT (US)

(72) Inventors: Daniel A. Gubler, Ph.D., Orem, UT (US); Tsz Ho Tsang, American Fork, UT (US)

(21) Appl. No.: 17/545,768

(22) Filed: Dec. 8, 2021

Related U.S. Application Data
(60) Provisional application No. 63/123,350, filed on Dec. 9, 2020.

(51) Int. Cl. A61K 31/575; A61K 31/4375; A61K 31/01; A61K 36/28; A61K 31/353; A61K 31/357; A61K 36/81; A61K 36/29; A61K 31/7034; A61K 36/15

(52) U.S. Cl. CPC A61K 31/575; A61K 31/4375; A61K 31/01; A61K 36/28; A61K 36/15; A61K 31/357; A61K 36/81; A61K 36/29; A61K 31/7034; A61K 31/353

(57) ABSTRACT

Elevated cholesterol levels, especially low lipoprotein cholesterol (LDL-C), leads to atherosclerosis—which may increase the risk of heart attack, stroke, and peripheral artery disease. Statins are common medications that can help lower cholesterol levels. However, myopathy can be a serious side effect that prevents patients from taking statins and significantly decreases overall quality of life. Described herein are dosage forms containing select combinations of natural herbal extracts and phytonutrients having the surprising ability to lower LDL-C levels in amounts comparable to statin drugs without noted side effects such as myopathy.

### CROSS-REFERENCE TO RELATED APPLICATION

This application claims the benefit under 35 U.S.C. § 119(e) of U.S. Provisional Patent Application Ser. No. 63/123,350, filed Dec. 9, 2020, the disclosure of which is hereby incorporated herein in its entirety by this reference.

### TECHNICAL FIELD

The application relates generally to nutritional supplements and associated methods of making and using them. More particularly, the application relates to a supplement containing multiple, select phytonutrients, which when used in combination, aid in, among other things, maintaining a subject's health through promoting healthy cholesterol levels.

### BACKGROUND

According to the CDC, more than 12% of adults age 20 and older in the United States have total cholesterol levels higher than 200 mg/dL. Carroll et al. (2017) infra. That number converts to nearly 29 million adults with high cholesterol. High blood cholesterol levels, especially elevated levels of LDL-C, significantly raise the risk for heart disease.

Different medications such as statins, fibrates, cholesterol absorption inhibitors, and bile acid sequestrants are used to combat elevated cholesterol levels. Statins drugs are the most commonly used options of these.

Statins, also known as HMG-CoA reductase inhibitors, are a class of lipid lowering medications. Such compounds inhibit enzyme HMG-CoA reductase, which plays a central role in the production of cholesterol. There are different chemical forms of statin drugs, such as atorvastatin, fluvastatin, lovastatin, rosuvastatin, and simvastatin. These statins are sold under different brand names for their ability to lower cholesterol—especially LDL-C. On average, statin drugs can lower a patient's LDL-C level by 1.8 mmol/L (70 mg/dL), which translates to a 17% reduced risk of stroke and an estimated 60% decrease in the number of cardiac events. Law et al. (2003) infra.

Unfortunately, side effects such as myopathy are not uncommon among statins users. It has been reported that 10-15% of statin users develop statin-related muscle side effects ranging from mild myalgia to severe muscle symptoms with significantly elevated creatine phosphokinase levels. Staffa et al. (2002) infra; Abd et al. (2011) infra. Statin-induced myopathy is higher with sporadic heavy exercise, in elderly patients, and those with a lower body mass index (“BMI”).

Different methods have been used to try to combat these unwanted side effects. For example, using a lower dose of statin, combining a lower dose of statin with other cholesterol lowering drugs such as ezetimibe or colesvalam, or using supplements such as red yeast rice (which have been banned by the FDA for use in dietary supplements and have been shown to contain impurities like cutrinin, which can cause kidney failure). Due to this background, new formulations that lower total and LDL-C cholesterol levels working through different modes of action are sorely needed to support human health and promote overall quality of life. In their 2015 CRN Consumer Survey on Dietary Supplements, the Council for Responsible Nutrition (“CRN”) found that 78% of Americans prefer taking dietary supplements over taking pharmaceutical drugs.

### BRIEF SUMMARY

Elevated cholesterol levels, especially low lipoprotein cholesterol (LDL-C), leads to atherosclerosis—which appears to increase the risk of heart attack, stroke, and peripheral artery disease. Statins are common medications that can help lower cholesterol levels. However, serious side effects such as myopathy can be seen, which prevents patients from taking statins and significantly decreases their overall quality of life. Described herein are dosage forms containing combinations of plant extracts and phytonutrients, which together have the ability to lower LDL-C levels to levels comparable with statin drugs. Also described herein is the use of non-naturally occurring combinations of selected phytonutrients that, when administered to (or consumed by) the subject, result in healthier cholesterol levels.

Specifically described is a dosage form for administration to a subject, the dosage form comprising at least five phytonutrients selected from the group consisting of phytosterol or phytosterol esters, berberine, lycopene, silymarin, polydatin, chitosan, danshen extract, Dioscorea nipponica Makia extract, golden kiwi root extract, olive extract, Scutellaria baicalensis extract, white mulberry extract, quercetin, Phaleria macrocarpa fruit extract, pinostrobin, and resveratrol.

Such a dosage form typically contains amounts of the selected at least five phytonutrients to promote healthy cholesterol levels in the subject when the subject ingests at least one of the dosage forms on a daily basis.

In certain embodiments, such a dosage form contains the selected at least five phytonutrients, when present, are present in the following amounts: from about 200 to about 9,000 mg of phytosterol or phytosterol esters, from about 200 to about 1,500 mg of berberine, from about 4 to about 500 mg of lycopene, from about 20 to about 1,000 mg of silymarin, from about 10 to about 1,000 mg of polydatin, from about 80 to about 1,500 mg of chitosan, from about 20 to about 1,000 mg of danshen extract, from about 10 to about 1,000 mg of Dioscorea nipponica Makia extract, from about 20 to about 1,000 mg of golden kiwi root extract, from about 10 to about 1,200 mg of olive extract, from about 20 to about 2,000 mg of Scutellaria baicalensis extract, from about 20 to about 1,000 mg of white mulberry extract, from about 50 to about 1,200 mg of quercetin, from about 20 to about 2,000 mg of Phaleria macrocarpa fruit extract, from about 20 to about 1,000 mg of pinostrobin, and from about 10 to about 1,000 mg of resveratrol. The contents of the dosage forms can be reduced appropriately when divided doses are contemplated (e.g., half the amount described if the dosage form is to be taken twice daily.) Taken daily, a dosage form containing such ingredients, at the amounts described, are able to inhibit proprotein convertase subtilisin/kexin-type 9 (“PCSK9”), downregulate PCSK9 gene, and/or inhibit cholesterol absorption, thus promoting lowering total cholesterol and LDL-C levels in a subject administered the dosage form.

In certain embodiments, the at least five phytonutrients are selected from the group consisting of phytosterol, berberine, lycopene, silymarin, and polydatin.

Such a dosage form is not natural or conventional and is typically in the form of a softgel, capsule, tablet, gel, powder, gummy, liquid, effervescent, bar, topical patch, serum, lotion, or cream. A method of making such dosage forms typically comprises admixing the selected phytonutrients with any appropriate filler(s) and excipient(s), and associating them together into or with the dosage form.

In certain embodiments, a method of maintaining a healthy cholesterol level in a subject comprises administering the dosage form to a subject in need thereof (e.g., someone diagnosed with undesirable cholesterol levels, or someone trying to prevent the occurrence of such levels).

In certain embodiments, a method as described herein comprises administering a combination of at least five selected phytonutrients to the subject, wherein the at least five phytonutrients are selected are from the group consisting phytosterol or phytosterol esters, berberine, lycopene, silymarin, polydatin, chitosan, danshen extract, Dioscorea nipponica Makia extract, golden kiwi root extract, olive extract, Scutellaria baicalensis extract, white mulberry extract, quercetin, Phaleria macrocarpa fruit extract, pinostrobin, and resveratrol.

With such methods, the subject typically ingests the selected at least five phytonutrients, when selected, in the following amounts on a daily basis: from about 200 to about 9,000 mg of phytosterol or phytosterol esters, from about 200 to about 1,500 mg of berberine, from about 4 to about 500 mg of lycopene, from about 20 to about 1,000 mg of silymarin, from about 10 to about 1,000 mg of polydatin, from about 80 to about 1,500 mg of chitosan, from about 20 to about 1,000 mg of danshen extract, from about 10 to about 1,000 mg of Dioscorea nipponica Makia extract, from about 20 to about 1,000 mg of golden kiwi root extract, from about 10 to about 1,200 mg of olive extract, from about 20 to about 2,000 mg of Scutellaria baicalensis extract, from about 20 to about 1,000 mg of white mulberry extract, from about 50 to about 1,200 mg of quercetin, from about 20 to about 2,000 mg of Phaleria macrocarpa fruit extract, from about 20 to about 1,000 mg of pinostrobin, and from about 10 to about 1,000 mg of resveratrol.

Such dosage forms may be administered in either single or divided dosages. For single dosages, they are typically administered once a day. For divided dosages, they are typically administered two or three times a day (e.g., with meals). In certain embodiments, dosage forms are taken by the subject about 15 minutes before a meal.

The dosage forms and/or methods described herein may be used for inhibiting PCSK9 and/or downregulating the PCSK9 gene and/or for maintaining a healthy cholesterol level or healthy cholesterol levels in a subject (e.g., a mammal, such as a human.) In certain embodiments, the described combinations are for use in lowering LDL-cholesterol in a subject. In certain embodiments, the described combinations are for use in the treatment of hypercholesterolemia. In certain embodiments, the described combinations are for use in the treatment of high levels of C-reactive protein (CRP) (e.g., levels greater than 10 mg/L.)

### DETAILED DESCRIPTION

Described is a selected combination of plant-natural products with the ability to lower cholesterol, LDL-cholesterol, LDL-C/HDL-C, and total cholesterol/HDL-C, while not lowering desirable HDL-cholesterol levels in the subject. The described compositions containing the selected combination are shown herein to unexpectedly lower total and LDL cholesterol levels in a subject substantially better than the sum of the components of the combination. These results establish an improvement over other dietary supplements and—at least with respect to lowering undesirable cholesterol levels—in efficacy comparable to statin drugs, which is significantly more than the individual components of the selected combinations. Further, as described herein, the described formulations should present an attractive alternative to prescription drugs since, unlike statins, they were not seen to lead to side effects such as myopathy.

Specifically described are compositions containing select different botanical extracts and phytonutrients with the ability to lower both total and LDL-C cholesterol levels. While not intending to be bound by theory, the following may help explain the surprising and unexpected results described herein. The described composition are found to inhibit proprotein convertase subtilisin/kexin-type 9 (“PCSK9”) and down-regulate the PCSK9 gene. PCSK9 is a secreted serine protease that regulates the post-transcriptional degradation of the LDL receptor to reduce cholesterol uptake. He et al. (2017) infra. PCSK9 binds directly to the epidermal growth factor repeat A (EGF-A) of the LDL receptor and takes part in the process by which LDL receptors are transported from endosomes to the lysosome for degradation. Cameron et al. (2008) infra. LDL receptors are crucial for regulating cholesterol level. LDL receptors transport LDL-cholesterol from blood into the cell so LDL-cholesterol can be degraded. The presence of PCSK9 in blood serum leads to the degradation of LDL receptor thus causing high cholesterol level in blood. The compositions described herein contain ingredients that suppress PCSK9—thus preventing degradation of the LDL receptor, while maintaining healthy cholesterol levels.

As previously identified herein, the dosage form comprise at least five phytonutrients selected from the group consisting of phytosterol or phytosterol esters, berberine, lycopene, silymarin, polydatin, chitosan, danshen extract, Dioscorea nipponica Makia extract, golden kiwi root extract, olive extract, Scutellaria baicalensis extract, white mulberry extract, quercetin, Phaleria macrocarpa fruit extract, pinostrobin, and resveratrol. Preferably, the at least five phytonutrients include at least one of phytosterol, berberine, lycopene, silymarin, and polydatin.

Phytosterol and/or phytosterol esters (sometimes called plant sterol and stanol esters) are readily commercially available. Methods of using the dosage forms described herein typically administer to the subject (on a daily basis) from about (plus or minus 5%) 200 mg to about 9,000 mg of phytosterol and/or phytosterol esters. Preferred daily dosages are between 1 and 2 grams. The administration of phytosterol and/or phytosterol esters (in amounts of 1.3 to 1.5 g daily) have been described as reducing LDL-cholesterol in subjects, but only in amounts of about 10.2%. See, e.g., Reaver et al. Nutrients (2019), 1, 2108; and Acuff et al. Lipids in Health and Disease (2007), 6:11.

Berberine is readily commercially available. Methods of using the dosage forms described herein typically administer to the subject (on a daily basis) from about (plus or minus 5%) 200 mg to about 1,500 mg of berberine. Preferred daily dosages are between 500 to 900 mg. The administration of berberine (in amounts of 1 g daily) have been described as reducing total cholesterol and LDL-cholesterol in subjects, but only in amounts of about 11.6% and 16.4% respectively. See, e.g., Derosa et al. Expert Opin. Biol. Ther. (2013), 13, 475-482. See, also, Galletti et al. Lipids in Health and Disease, (2019) 2, 66; and Sola et al. PLOS one, (2014) 9, e101978.

Lycopene is readily commercially available. Methods of using the dosage forms described herein typically administer to the subject (on a daily basis) from about (plus or minus 5%) 4 mg to about 500 mg of lycopene. Preferred daily dosages are between 100 to 250 mg. The administration of lycopene (in amounts of 22 mg daily) have been described as reducing LDL-cholesterol in subjects, but only in amounts of about 3.7 mg/dl. See, e.g., Nishimura et al. Nutrients (2019) 11, nu11051177. See, also, Misra et al. J. Obstet. Gynaecol. Res. (2006) 32, 299-304 (for a composition containing 4 mg lycopene, together with vitamin A, vitamin E, zinc sulfate monohydrate, and monohydrated selenium dioxide, which reportedly lowered both total cholesterol and LDL-cholesterol in subjects).

Silymarin is readily commercially available. Methods of using the dosage forms described herein typically administer to the subject (on a daily basis) from about (plus or minus 5%) 20 mg to about 1,000 mg of silymarin. Preferred daily dosages are between 250 to 750 mg. Silymarin is the extract of Silybum marianum, or milk thistle, and its major active compound is silybin. The administration of silymarin (in amounts of 600 mg daily) have been described as reducing total cholesterol and LDL-cholesterol in subjects, but only in amounts of about 27 mg/dl and 17 mg/dl respectively. See, e.g., Huseini et al. Phytotherapy Research (2006) 20, 1036-1039.

Polydatin is readily commercially available. Methods of using the dosage forms described herein typically administer to the subject (on a daily basis) from about (plus or minus 5%) 10 mg to about 1,000 mg of polydatin. Preferred daily dosages are between 250 to 750 mg.

Chitosan is readily commercially available. Methods of using the dosage forms described herein typically administer to the subject (on a daily basis) from about (plus or minus 5%) 80 mg to about 1,500 mg of chitosan.

Danshen extract is readily commercially available. Methods of using the dosage forms described herein typically administer to the subject (on a daily basis) from about (plus or minus 5%) 20 mg to about 1,000 mg of danshen extract. Preferred daily dosages are between 250 to 750 mg.

Dioscorea nipponica Makia extract is readily commercially available. Methods of using the dosage forms described herein typically administer to the subject (on a daily basis) from about (plus or minus 5%) 10 mg to about 1,000 mg of the extract. Preferred daily dosages are between 250 to 750 mg.

Golden kiwi root extract is readily commercially available. Methods of using the dosage forms described herein typically administer to the subject (on a daily basis) from about (plus or minus 5%) 20 mg to about 1,000 mg of golden kiwi root extract. Preferred daily dosages are between 250 to 750 mg.

Olive extract is readily commercially available. Methods of using the dosage forms described herein typically administer to the subject (on a daily basis) from about (plus or minus 5%) 10 mg to about 1,200 mg of the extract. Preferred daily dosages are between 250 to 750 mg.

Scutellaria baicalensis extract is readily commercially available. Methods of using the dosage forms described herein typically administer to the subject (on a daily basis) from about (plus or minus 5%) 20 mg to about 2,000 mg of the extract. Preferred daily dosages are between 250 to 1,000 mg.

White Mulberry extract is readily commercially available. Methods of using the dosage forms described herein typically administer to the subject (on a daily basis) from about (plus or minus 5%) 20 mg to about 1,000 mg of white mulberry extract. Preferred daily dosages are between 250 to 750 mg.

Quercetin is readily commercially available. Methods of using the dosage forms described herein typically administer to the subject (on a daily basis) from about (plus or minus 5%) 50 mg to about 1,200 mg of quercetin. Preferred daily dosages are between 250 to 750 mg.

Phaleria macrocarpa fruit extract is readily commercially available. Methods of using the dosage forms described herein typically administer to the subject (on a daily basis) from about (plus or minus 5%) 20 mg to about 2,000 mg of the extract. Preferred daily dosages are between 250 to 1,000 mg.

Pinostrobin is readily commercially available. Methods of using the dosage forms described herein typically administer to the subject (on a daily basis) from about (plus or minus 5%) 20 mg to about 1,000 mg of pinostrobin. Preferred daily dosages are between 250 to 750 mg.

Resveratrol is readily commercially available. Methods of using the dosage forms described herein typically administer to the subject (on a daily basis) from about (plus or minus 5%) 10 mg to about 1,000 mg of resveratrol. Preferred daily dosages are between 250 to 750 mg. The administration of resveratrol (in amounts of 250 mg daily) have been described as reducing total cholesterol and LDL-cholesterol in subjects, but only in amounts of about 29 mg/dl and 19.7 mg/dl respectively. See, e.g., Batista-Jorge et al. Life Science (2020) 256, 117962.

In certain embodiments, the methods are particularly useful in treating patients with, for example, high levels of C-reactive protein (“CRP”), since the described compositions do not appear to significantly raise HDL cholesterol in the subject, which has been identified as risky for them. See, e.g., R. Dullaart “Increased Coronary Heart Disease Risk Determined by High High-Density Lipoprotein Cholesterol and C-Reactive Protein: Modulation by Variation in the CETP Gene” Arteriosclerosis, Thrombosis, and Vascular Biology, 2010; 30:1502-1503. Typically, levels of C-reactive protein (CRP) greater than 10 mg/L are considered as a “high level.” Thus, the methods described herein may be particularly useful, when the subject being administered the dosage form(s) has relatively high levels of HDL cholesterol and C-reactive protein (CRP) as may be determined by standard blood testing in comparison to a healthy subject.

Once being apprised of the instant disclosure, a person of ordinary skill in the art will be readily able to make or prepare the dosage forms using Galenical techniques. Preferably, a finished product delivery forms is selected from the group consisting of a softgel, capsule, tablet, gel, powder, gummy, liquid, effervescent, bar, topical patch, serum, lotion, and cream.

Furthermore, the described dosage forms and methods can be used in combination with other cholesterol-lowering therapies, such as the administration of statins.

The invention is further described with the aid of the following illustrative Examples.

### Example I

More than 112 dosage forms containing 570 mg phytosterol, 440 mg berberine, 52.5 mg lycopene, 240 mg silymarin and 40.5 mg polydatin were made.

### Example II

A pilot study was conducted with four people. Study subjects were instructed to fast for 12 hours before taking blood measurements occurring at time 0 and 4 weeks after supplementation with the formula of Example I once a day before their largest meal of the day.

Triglyceride, total cholesterol, and HDL-cholesterol were measured using Cardiochek Home Use Analyzer from pts Diagnostics of Whitestown, Ind., US. LDL-cholesterol was calculated using the Friedewald Equation.

Participants were given 4 week servings of Composition of Example I and, after their blood test at time 0, were instructed to take the supplement 15 minutes before their biggest meal daily and for 28 consecutive days thereafter. Participants were instructed to maintain their normal diet and physical activities. A final blood test was conducted on the 31st 29th day after daily consumption of the composition of Example I.

Results: After 4 weeks of supplementation with the composition of Example I, there was a significant decrease in total cholesterol (−87.38 mg/dL (−40%), p<0.05) and LDL-cholesterol (−75.15 mg/dL (−54%), p<0.05). There was also a decrease in triglyceride level (−48.63 mg/dL), but the result was not statically significant. There was a decrease in LDL-C/HDL-C (−1.48, p<0.05) and total cholesterol/HDL-C (−1.91, p<0.05). HDL-C did not see any significant change after 4 weeks of supplementation.

Daily consumption of the composition of Example I yielded a drastic decrease in total cholesterol and LDL-cholesterol levels in comparison to other cholesterol-lowering supplements containing plant phytonutrients and other vitamins/minerals without altering HDL-cholesterol levels. Surprisingly, Composition of Example I reduced total and LDL cholesterol in a period of four weeks—which is dramatically shorter than the 8-24 weeks often required to see an effect in other cholesterol lowering supplements. See, Galletti et al. (2019); Sola et al. (2014); Derosa et al. (2013); Reaver et al. (2019); Acuff et al (2007); Nishimura et al. (2019); Misra et al. (2006); Huseini et al. (2006); Batista-Jorge et al. (2020); and Sprecher et al. (2002).

Not only did the composition of Example I have astonishing results compared against other cholesterol lowering dietary supplements, this study found that the cholesterol-lowering ability of the composition of Example I showed comparable results to statin pharmaceuticals.

Table 1 shows the reduction of serum LDL cholesterol levels (mg/dL) according to daily oral doses of different statin drugs. Law et al. (2003) infra screened 165 different clinical studies on different statins with various dosages. These studies lasted typically a few weeks. The results of these studies were compared against the composition of Example I. Only rosuvastatin showed a greater cholesterol-lowering effect at 20 mg or above than the composition of Example I.

TABLE 1
Reduction of LDL-C levels (mg/dL) of different statins
oral dosage vs Composition of Example I.

| | Rreduction in LDL-C levels (mg/dL) | | | | |
|---|---|---|---|---|---|
| | 5 mg | 10 mg | 20 mg | 40 mg | 80 mg |
| Atorvastatin | 58.3917 | 69.2193 | 80.0469 | 91.2612 | 102.0888 |
| Fluvastatin | 17.7882 | 28.6158 | 39.4434 | 50.271 | 61.0986 |
| Lovastatin | NA | 39.4434 | 54.138 | 68.4459 | 83.1405 |
| Pravastatin | 28.2291 | 36.7365 | 45.2439 | 53.3646 | 61.872 |
| Rosuvastatin | 71.1528 | 80.4336 | 89.7144 | 98.9952 | 108.276 |
| Simvastatin | 41.7636 | 50.6577 | 59.5518 | 68.8326 | 77.7267 |

| | |
|---|---|
| Formula A | 75.15 |

Table 2 also shows that the composition of Example I provides LDL-cholesterol reduction results comparable to other statins used at clinically relevant dosages. With a 54% lowering of LDL-C levels, the composition of Example I outperformed all statins besides rosuvastatin (80 mg) and atorvastatin (20 mg and above). Fan et al. (2020) infra. With a price of about $59/month, the composition of Example I can be much less expensive than branded statin drugs which, depending on dosage, range anywhere from $44-US $508/month.

TABLE 2
Statin Dose Intensity and price vs Composition of Example I

| | | Treatment Options | | | | | | | |
|---|---|---|---|---|---|---|---|---|---|
| | % LDL-C | | Crestor | Lipitor | Livalo | Zocor | Alloprev 24-hour | Pravachol | Lescol XL |
| Intensity | Reduction | Formula A | (Rosuvastatin) | (Atorvastatin) | (Pitavastatin) | (Simvastatin) | (Lovastatin) | (Pravastatin) | (Fluvastatin) |
| High | 63 | | 40 mg, | | | | | | |
| Intensity | 62 | | $242/mo | | | | | | |
| (lowers | 61 | | | | | | | | |
| LDL-C < | 60 | | | 80 mg, | | | | | |
| 50%) | 59 | | | $508/mo | | | | | |
| | 58 | | | | | | | | |
| | 56 | | 20 mg, | | | | | | |
| | 54 | $60/mo | $242/mo | | | | | | |
| | 52 | | 10 mg, | 40 mg, | | | | | |
| | 50 | | $242/mo | $508/mo | | | | | |
| Moderate- | 48 | | | | | | | | |
| Intensity | 46 | | | | | | | | |
| (lowers | 44 | | 5 mg, | | | | | | |
| LDL-C | 42 | | $242/mo | 20 mg, | 4 mg, | | | | |
| 90-49%) | 40 | | | $508/mo | $293/mo | 40 mg, | | | |
| | 38 | | | 10 mg, | | $261/mo | 80 mg, | | |
| | 36 | | | $356/mo | | | $263/mo | 80 mg, | |
| | 34 | | | | | 20 mg, | | $367/mo | 80 mg, |
| | 32 | | | | 2 mg, | $261/mo | | 40 mg, | $350/mo |
| | | | | | $293/mo | | | $132/mo | |
| | 30 | | | | 1 mg, | 10 mg, | 40 mg, | 20 mg, | |
| Low- | 28 | | | | $293 mo | $150/mo | $132/mo | $125/mo | |
| Intensity | 26 | | | | | 5 mg, | 20 mg, | | |
| (lowers | 24 | | | | | $75/mo | $79/mo | | 40 mg, |
| LDL-C < | 22 | | | | | | | | $175/mo |
| 30%) | 20 | | | | | | 10 mg, | 10 mg, | 20 mg, |
| | 18 | | | | | | $44/mo | $63/mo | $88/mo |

In conclusion, a formula that contains the ingredients as described herein (e.g., the composition of Example I) can perform better than other cholesterol lowering supplements. The described dosage forms achieve similar or even better results than most statins medications, but were not seen to lead to side effects such as myopathy.

### Example III

Dosage forms each containing the following are made:

200 mg of Scutellaria baicalensis extract,

200 mg of white mulberry extract,

100 mg of quercetin,

100 mg of Phaleria macrocarpa fruit extract,

100 mg of pinostrobin, and

75 mg of resveratrol.

### Example IV

Capsules each containing the following are made:

50 mg of lycopene,

300 mg of silymarin,

50 mg of polydatin,

10 mg of chitosan, and

150 mg of resveratrol.

### Example V

Tablets each containing the following are made:

250 mg of lycopene,

100 mg of silymarin,

100 mg of polydatin,

100 mg of danshen extract, and

100 mg of quercetin.

### REFERENCES

(The contents of each of which are incorporated herein by this reference.)
Abd, T. T. et al “Statin induced myopathy: a review and update” Expert Opinion Drug Safety, 2011, 10, 373-387.
Acuff et al. Lipids in Health and Disease (2007), 6:11.
Cameron, J. et al “Berberine decreases PCSK9 expression in HepG2 cells” Atherosclerosis, 2008, 201, 266-273.
Carroll M, Fryar C, Nguyen D. HDL, National Health and Nutrition Examination Survey: Total and High-density Lipoprotein Cholesterol in Adults: United States, 2015-2016. NCHS data brief, no. 290. Hyattsville, Md.: National Center for Health Statistics; 2017.
Derosa et al. Expert Opin. Biol. Ther. (2013), 13, 475-482.
R. Dullaart “Increased Coronary Heart Disease Risk Determined by High High-Density Lipoprotein Cholesterol and C-Reactive Protein: Modulation by Variation in the CETP Gene” Arteriosclerosis, Thrombosis, and Vascular Biology, 2010; 30:1502-1503.
Fan, A. L. et al. “Screening and Management of Lipids” UMHS Lipid Therapy Guideline, 2020.
Galletti et al. Lipids in Health and Disease, (2019) 2, 66.
He, M. et al “Actinidia chinensis Planch root extract inhibits cholesterol metabolism in hepatocellular carcinoma through upregulation of PCSK9” Oncotarget, 2017, 8, 42136-42148.
Huseini et al. Phytotherapy Research (2006) 20, 1036-1039.
Law, M. R. et al “Quantifying effect of statins on low density lipoprotein cholesterol, ischaemic heart disease, and stroke: systematic review and meta-analysis” BMJ, 2003, 326, 1423-1427.
Misra et al. J. Obstet. Gynaecol. Res. (2006) 32, 299-304.
Nishimura et al. Nutrients (2019) 11, nu11051177.
Reaver et al. Nutrients (2019), 1, 2108.
Sola et al. PLOS one, (2014) 9, e101978.
Sprecher et al. Metabolism (2002) 51, 1166-1170.
Staffa, J. A. et al “Cerivastatin and Reports of Fatal Rhabdomyolysis” N Engl J Med, 2002, 346, 539.
2015 CRN Consumer Survey on Dietary Supplements; https://www.crnusa.org/CRN-consumersurvey-archives/2015/.

1. A dosage form for administration to a subject, the dosage form comprising at least five phytonutrients selected from the group consisting of phytosterol or phytosterol esters, berberine, lycopene, silymarin, polydatin, chitosan, danshen extract, Dioscorea nipponica Makia extract, golden kiwi root extract, olive extract, Scutellaria baicalensis extract, white mulberry extract, quercetin, Phaleria macrocarpa fruit extract, pinostrobin, and resveratrol.

2. The dosage form of claim 1, wherein the dosage form contains amounts of the selected at least five phytonutrients to promote healthy cholesterol levels in the subject when the subject ingests at least one of the dosage forms on a daily basis.

3. The dosage form of claim 1, wherein the selected at least five phytonutrients, when present, are present in the following amounts:
from about 200 to about 9,000 mg of phytosterol or phytosterol esters,
from about 200 to about 1,500 mg of berberine,
from about 4 to about 500 mg of lycopene,
from about 20 to about 1,000 mg of silymarin,
from about 10 to about 1,000 mg of polydatin,
from about 80 to about 1,500 mg of chitosan,
from about 20 to about 1,000 mg of danshen extract,
from about 10 to about 1,000 mg of Dioscorea nipponica Makia extract,
from about 20 to about 1,000 mg of golden kiwi root extract,
from about 10 to about 1,200 mg of olive extract,
from about 20 to about 2,000 mg of Scutellaria baicalensis extract,
from about 20 to about 1,000 mg of white mulberry extract,
from about 50 to about 1,200 mg of quercetin,
from about 20 to about 2,000 mg of Phaleria macrocarpa fruit extract,
from about 20 to about 1,000 mg of pinostrobin, and
from about 10 to about 1,000 mg of resveratrol.

4. The dosage form of claim 3, wherein the at least five phytonutrients are selected from the group consisting of phytosterol, berberine, lycopene, silymarin, and polydatin.

5. The dosage form of claim 3, wherein the dosage form is selected from the group consisting of a softgel, capsule, tablet, gel, powder, gummy, liquid, effervescent, bar, topical patch, serum, lotion, and cream.

6. A method of inhibiting PCSK9 and/or downregulating the PCSK9 gene in a subject, the method comprising:
administering the dosage form of claim 1 to the subject in an amount so as to inhibit PCSK9 and/or downregulate the PCSK9 gene in the subject.

7. A method of maintaining a healthy cholesterol level or healthy cholesterol levels in a subject, the method comprising:
administering the dosage form of claim 2 to the subject so as to maintain a healthy cholesterol level or healthy cholesterol levels in the subject.

8. A method of treating hypercholesterolemia in a subject, the method comprising:
administering the dosage form of claim 2 to the subject so as to treat hypercholesterolemia in the subject.

9. A method of reducing C-reactive protein (CRP) levels greater than 10 mg/L in a subject, the method comprising:
administering the dosage form of claim 1 to the subject in an amount so as to reduce levels of CRP greater than 10 mg/L in the subject.

10. A method of maintaining a healthy cholesterol level in a subject, the method comprising:
administering the dosage form of claim 2 to the subject.

11. A method of maintaining a healthy cholesterol level in a subject, the method comprising:
administering a combination of at least five selected phytonutrients to the subject, wherein the at least five phytonutrients are selected are from the group consisting phytosterol or phytosterol esters, berberine, lycopene, silymarin, polydatin, chitosan, danshen extract, Dioscorea nipponica Makia extract, golden kiwi root extract, olive extract, Scutellaria baicalensis extract, white mulberry extract, quercetin, Phaleria macrocarpa fruit extract, pinostrobin, and resveratrol.

12. The method according to claim 10 or claim 11, wherein the subject ingests the selected at least five phytonutrients, when selected, in the following amounts on a daily basis:
from about 200 to about 9,000 mg of phytosterol or phytosterol esters,
from about 200 to about 1,500 mg of berberine,
from about 4 to about 500 mg of lycopene,
from about 20 to about 1,000 mg of silymarin,
from about 10 to about 1,000 mg of polydatin,
from about 80 to about 1,500 mg of chitosan,
from about 20 to about 1,000 mg of danshen extract,
from about 10 to about 1,000 mg of Dioscorea nipponica Makia extract,
from about 20 to about 1,000 mg of golden kiwi root extract,
from about 10 to about 1,200 mg of olive extract,
from about 20 to about 2,000 mg of Scutellaria baicalensis extract,
from about 20 to about 1,000 mg of white mulberry extract,
from about 50 to about 1,200 mg of quercetin,
from about 20 to about 2,000 mg of Phaleria macrocarpa fruit extract,
from about 20 to about 1,000 mg of pinostrobin, and
from about 10 to about 1,000 mg of resveratrol.

13. The method according to claim 11, wherein the at least five phytonutrients comprise phytosterol, berberine, lycopene, silymarin, and polydatin.

14. The method according to claim 10, wherein PCSK9 is inhibited and/or the PCSK9 gene is downregulated in the subject.

15. The method according to claim 10, wherein the subject has relatively high levels of HDL cholesterol and C-reactive protein (CRP) as may be determined by standard blood testing in comparison to a healthy subject.

16. (canceled)

17. The method according to claim 11, wherein the subject ingests the selected at least five phytonutrients, when selected, in the following amounts on a daily basis:
from about 200 to about 9,000 mg of phytosterol or phytosterol esters,
from about 200 to about 1,500 mg of berberine,
from about 4 to about 500 mg of lycopene,
from about 20 to about 1,000 mg of silymarin,
from about 10 to about 1,000 mg of polydatin,
from about 80 to about 1,500 mg of chitosan,
from about 20 to about 1,000 mg of danshen extract,
from about 10 to about 1,000 mg of Dioscorea nipponica Makia extract,
from about 20 to about 1,000 mg of golden kiwi root extract,
from about 10 to about 1,200 mg of olive extract,
from about 20 to about 2,000 mg of Scutellaria baicalensis extract,
from about 20 to about 1,000 mg of white mulberry extract,
from about 50 to about 1,200 mg of quercetin,
from about 20 to about 2,000 mg of Phaleria macrocarpa fruit extract,
from about 20 to about 1,000 mg of pinostrobin, and
from about 10 to about 1,000 mg of resveratrol.

18. The method according to claim 17, wherein the at least five phytonutrients comprise phytosterol, berberine, lycopene, silymarin, and polydatin.

19. The method according to claim 17, wherein PCSK9 is inhibited and/or the PCSK9 gene is downregulated in the subject.

20. The method according to claim 11, wherein PCSK9 is inhibited and/or the PCSK9 gene is downregulated in the subject.

21. The method according to claim 11, wherein the subject has relatively high levels of HDL cholesterol and C-reactive protein (CRP) as may be determined by standard blood testing in comparison to a healthy subject.
